(19) United States
(12) Patent Application Publication
(10) Pub. No.: US 2014/0058263 A1
(43) Pub. Date: Feb. 27, 2014

(54) Adaptive Ultrasonic Array

(71) Applicants: Michael H. Baym, Cambridge, MA (US); Roderick A. Hyde, Redmond, WA (US); Jordin T. Kare, Seattle, WA (US); Lowell L. Wood, JR., Bellevue, WA (US)

(72) Inventors: Michael H. Baym, Cambridge, MA (US); Roderick A. Hyde, Redmond, WA (US); Jordin T. Kare, Seattle, WA (US); Lowell L. Wood, JR., Bellevue, WA (US)

(73) Assignee: Elwha LLC, a limited liability company of the State of Delaware

(21) Appl. No.: 13/593,789

(22) Filed: Aug. 24, 2012

Related U.S. Application Data
(63) Continuation-in-part of application No. 13/593,804, filed on Aug. 24, 2012.

(51) Int. Cl. A61B 8/00; A61B 8/08; A61B 8/14

(52) U.S. Cl. 600/447; 600/459; 600/453

(57) ABSTRACT

An ultrasound array uses information about contact quality with the body to weight produced ultrasound data.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications, including any priority claims, is incorporated herein by reference to the extent such subject matter is not inconsistent herewith.

### RELATED APPLICATIONS

For purposes of the USPTO extra-statutory requirements, the present application constitutes a continuation-in-part of United States Patent Application No. TO BE ASSIGNED, entitled ADAPTIVE ULTRASONIC ARRAY, naming Michael H. Baym, Roderick A. Hyde, Jordin T. Kare, and Lowell L. Wood, Jr. as inventors, filed 24 Aug. 2012, which is currently co-pending or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above, but expressly points out that such designation(s) are not to be construed in any way as any type of commentary and/or admission as to whether or not the present application contains any new matter in addition to the matter of its parent application(s).

### SUMMARY

In one aspect, an ultrasound array includes a plurality of transducer elements configured for contact with a body, and a controller configured to determine a quality of acoustic coupling with the body at each transducer element and to use the determined coupling qualities to apply a weighting to ultrasonic data received from the plurality of transducer elements. The array may further include one or more ultrasound source(s), which may be associated with particular transducer elements. The transducer elements may be configured for contact with a living body or a human body, or they may be incorporated into a garment. Determining coupling quality may include determining magnitude of an echo or reflection from an exterior surface of the body or determining magnitude of a signal passing through the body, and may include determining coupling quality at a different frequency from ultrasound imaging. The array may further include a display configured to display an ultrasound image. Applying a weighting may include using ultrasonic data from a subset of the transducers, for example discarding null signals from transducers having a relatively poor contact quality, and may include using contact information from other transducers to determine the weight given to a signal from one or more transducers. Applying a weighting may include determining whether an image of a given quality can be produced, and may further include communicating the fact to a user if it cannot or producing an image only after determining that it can. The transducer elements may be configured to measure a Doppler shift frequency. The array may be configured to respond to determined quality of acoustic coupling by adjusting a quantity of coupling fluid or by adjusting a contact force of a transducer. Determining quality of acoustic coupling may include determining the quality at a succession of points in time, and applying a weighting may further include applying the weighting at the succession of points in time.

In another aspect, an ultrasound array includes a plurality of ultrasound sources configured for contact with a body, a plurality of ultrasound receivers configured to receive ultrasound from the plurality of sources, and a controller configured to determine a quality of acoustic coupling with the body at each ultrasound source and to use the determined qualities to apply a weighting to a power level of each of the plurality of ultrasound sources. Applying a weighting may include determining an in-contact array pattern for the ultrasound sources, and using the determined pattern to calculate a phase-intensity distribution profile selected to deliver a selected irradiation distribution in the body. Applying a weighting may include adjusting a power level of at least some of the ultrasound sources, for example by increasing power to a source having better coupling with the body, increasing power to a source having worse coupling with the body, deactivating a source having inferior coupling, or maintaining a constant total energy of ultrasound coupled into the body by some or all of the ultrasound sources. The ultrasound sources may be configured for contact with a living body or a human body, or they may be incorporated into a garment. Determining coupling quality may include determining magnitude of an echo or reflection from an exterior surface of the body or determining magnitude of a signal passing through the body, and may include determining coupling quality at a different frequency from ultrasound imaging. The array may further include a display configured to display an ultrasound image. Applying a weighting may include using ultrasonic data from a subset of the receivers, for example discarding null signals from receivers having a relatively poor contact quality. Applying a weighting may include determining whether an image of a given quality can be produced, and may further include communicating the fact to a user if it cannot or producing an image only after determining that it can. The ultrasound receivers may be configured to measure a Doppler shift frequency. The array may be configured to respond to determined quality of acoustic coupling by adjusting a quantity of coupling fluid or by adjusting a contact force of a receiver or source. Determining quality of acoustic coupling may include determining the quality at a succession of points in time, and applying a weighting may further include applying the weighting at a succession of points in time.

In another aspect, an ultrasound method includes applying a plurality of ultrasonic transducer elements to a body, determining a quality of acoustic coupling with the body at each transducer element, and using the determined qualities to apply a weighting to ultrasonic data generated by each transducer element. The method may further include applying one or more ultrasound source(s) to the body, which may be a living body or a human body. The method may further include adjusting a power level of a transducer in response to acoustic coupling quality, for example by turning off the transducer, or by increasing power to a transducer having superior (or inferior) coupling quality. Applying a weighting to the ultrasonic data may include defining an in-contact array pattern, and using the defined pattern to calculate a phase-intensity distribution profile in order to achieve a selected irradiation distribution in the body, and may further include applying the phase-intensity distribution profile. Applying a weighting may include ignoring null signals from out-of-contact transducer elements. Determining a quality of acoustic coupling may include measuring acoustic coupling at a first frequency, and gathering ultrasonic data at a second different frequency. The method may further include displaying an ultrasound image. Applying a weighting may include using data only from a subset of transducer elements, and may include adjusting power level(s) of only a subset of transducer elements, for example by increasing power to an element having a high quality of acoustic coupling with the body, removing power to an element having a low coupling quality, or maintaining a constant total power of ultrasound. Applying a weighting may include discarding null signals from transducers having a relatively poor quality of acoustic coupling with the body. Applying a weighting may include determining whether a sufficient number of transducers having a sufficient quality of acoustic coupling to generate an ultrasound image of a selected quality, and may further include signaling that the selected quality cannot be achieved, adjusting a quantity of coupling fluid, or adjusting a contact force. Determining a quality of acoustic coupling may include determining the quality at a succession of points in time, and applying a weighting may include applying the weighting to ultrasonic data captured in the vicinity of each of these points in time.

In another aspect, an ultrasound method includes applying a plurality of ultrasound sources to a body (e.g., a living body or a human body), applying a plurality of ultrasound receivers to the body, the receivers being configured to receive ultrasound from at least one of the sources, determining a quality of acoustic coupling with the body at each ultrasound source, and using the determined qualities of acoustic coupling to apply a weighting to a power level of each of the ultrasound sources. Applying a weighting to the ultrasonic data may include defining an in-contact array pattern, and using the defined pattern to calculate a phase-intensity distribution profile in order to achieve a selected irradiation distribution in the body, and may further include applying the phase-intensity distribution profile. Applying a weighting may include deactivating at least one ultrasound source, and may include adjusting power level(s) of only a subset of ultrasound sources, for example by increasing power to a source having a high (or low) quality of acoustic coupling with the body, removing power to a source having a low coupling quality, or maintaining a constant total power of ultrasound. Determining a quality of acoustic coupling may include measuring acoustic coupling at a first frequency, and gathering ultrasonic data at a second different frequency. The method may further include displaying an ultrasound image. Applying a weighting may include determining whether a sufficient number of transducers having a sufficient quality of acoustic coupling to generate an ultrasound image of a selected quality, and may further include signaling that the selected quality cannot be achieved. Determining a quality of acoustic coupling may include determining the quality at a succession of points in time, and applying a weighting may include applying the weighting to ultrasonic data captured in the vicinity of each of these points in time.

In another aspect, an ultrasound array includes a plurality of transducer elements configured for contact with a body, and a controller configured to determine a quality of acoustic coupling with the body at each transducer element and to use the determined qualities to deactivate transducer elements not meeting a threshold quality of acoustic coupling. The array may further include one or more ultrasound source(s), which may be associated with particular transducer elements. The transducer elements may be configured for contact with a living body or a human body, or they may be incorporated into a garment. Determining coupling quality may include determining magnitude of an echo or reflection from an exterior surface of the body, or determining magnitude of a signal passing through the body. The array may further include a display configured to display an ultrasound image. The transducers may be configured to measure a Doppler shift frequency. Determining quality of acoustic coupling may include determining the quality at a succession of points in type, and applying a weighting may further include applying the weighting at a succession of points in time.

In another aspect, an ultrasound array includes a plurality of transducer elements configured for contact with a body, a plurality of contact sensors, each configured to determine a quality of acoustic coupling with the body at a selected transducer element, and a controller configured to accept quality determinations from the plurality of sensors and use the accepted quality determinations to apply a weighting to ultrasonic data received from the plurality of transducer elements. The contact sensors may be configured to measure contact force with the body, or an electrical property (e.g., resistance or capacitance). The array may further include one or more ultrasound source(s), which may be associated with particular transducer elements. The transducer elements or the contact sensors may be configured for contact with a living body or a human body, or they may be incorporated into a garment. The array may further include a display configured to display an ultrasound image. Applying a weighting may include using ultrasonic data from a subset of the transducers, for example discarding null signals from transducers having a relatively poor contact quality, and may include using contact information from other transducers to determine the weight given to a signal from one or more transducers. Applying a weighting may include determining whether an image of a given quality can be produced, and may further include communicating the fact to a user if it cannot or producing an image only after determining that it can. The transducer elements may be configured to measure a Doppler shift frequency. The array may be configured to respond to determined quality of acoustic coupling by adjusting a quantity of coupling fluid or by adjusting a contact force of a transducer. Determining quality of acoustic coupling may include determining the quality at a succession of points in time, and applying a weighting may further include applying the weighting at the succession of points in time.

In another aspect, an ultrasound array includes a plurality of ultrasound sources configured for contact with a body, a plurality of ultrasound receivers configured to receive ultrasound from the plurality of sources, a plurality of contact sensors, each configured to determine a quality of acoustic coupling with the body at a selected ultrasound source, and a controller configured to accept quality determinations from the contact sensors and to use the accepted quality determinations to apply a weighting to ultrasonic data received by the ultrasound receivers. The contact sensors may be configured to measure contact force with the body, or an electrical property (e.g., resistance or capacitance). Applying a weighting may include determining an in-contact array pattern for the ultrasound sources, and using the determined pattern to calculate a phase-intensity distribution profile selected to deliver a selected irradiation distribution in the body. Applying a weighting may include adjusting a power level of at least some of the ultrasound sources, for example by increasing power to a source having better (or worse) coupling with the body, deactivating a source having inferior coupling, or maintaining a constant total energy of ultrasound coupled into the body by some or all of the ultrasound sources. The ultrasound sources or contact sensors may be configured for contact with a living body or a human body, or they may be incorporated into a garment. Determining coupling quality may include determining magnitude of an echo or reflection from an exterior surface of the body, and may include determining coupling quality at a different frequency from ultrasound imaging. The array may further include a display configured to display an ultrasound image. Applying a weighting may include using ultrasonic data from a subset of the receivers, for example discarding null signals from receivers having a relatively poor contact quality. Applying a weighting may include determining whether an image of a given quality can be produced, and may further include communicating the fact to a user if it cannot or producing an image only after determining that it can. The transducer elements may be configured to measure a Doppler shift frequency. The array may be configured to respond to determined quality of acoustic coupling by adjusting a quantity of coupling fluid or by adjusting a contact force of a receiver or source. Determining quality of acoustic coupling may include determining the quality at a succession of points in type, and applying a weighting may further include applying the weighting at a succession of points in time.

In another aspect, an ultrasound method includes applying a plurality of ultrasonic transducer elements to a body, applying a plurality of contact sensors to the body, each configured to determine a quality of acoustic coupling with the body at a selected transducer element, determining a quality of acoustic coupling with the body at each transducer element, and using the determined qualities to apply a weighting to ultrasonic data generated by each transducer element. Determining a quality of acoustic coupling may include determining contact force with the body, or an electrical property (e.g., resistance or capacitance). The method may further include applying one or more ultrasound source(s) to the body, which may be a living body or a human body. The method may further include adjusting a power level of a transducer in response to acoustic coupling quality, for example by turning off the transducer. Applying a weighting to the ultrasonic data may include defining an in-contact array pattern, and using the defined pattern to calculate a phase-intensity distribution profile in order to achieve a selected irradiation distribution in the body, and may further include applying the phase-intensity distribution profile. Applying a weighting may include ignoring null signals from out-of-contact transducer elements. The method may further include displaying an ultrasound image. Applying a weighting may include using data only from a subset of transducer elements, and may include adjusting power level(s) of only a subset of transducer elements, for example by increasing power to an element having a high (or low) quality of acoustic coupling with the body, removing power to an element having a low coupling quality, or maintaining a constant total power of ultrasound. Applying a weighting may include discarding null signals from transducers having a relatively poor quality of acoustic coupling with the body. Applying a weighting may include determining whether a sufficient number of transducers having a sufficient quality of acoustic coupling to generate an ultrasound image of a selected quality, and may further include signaling that the selected quality cannot be achieved, adjusting a quantity of coupling fluid, or adjusting a contact force. Determining a quality of acoustic coupling may include determining the quality at a succession of points in time, and applying a weighting may include applying the weighting to ultrasonic data captured in the vicinity of each of these points in time.

In another aspect, an ultrasound method includes applying a plurality of ultrasound sources to a body (e.g., a living body or a human body), applying a plurality of ultrasound receivers to the body, the receivers configured to receive ultrasound from at least one of the sources, applying a plurality of contact sensors to the body, each configured to determine a quality of acoustic coupling with the body at an ultrasound source, determining a quality of acoustic coupling with the body at each ultrasound source, and using the determined qualities of acoustic coupling to apply a weighting to ultrasonic data generated by each ultrasonic receiver. Determining a quality of acoustic coupling may include determining contact force with the body, or an electrical property (e.g., resistance or capacitance). Applying a weighting to the ultrasonic data may include defining an in-contact array pattern, and using the defined pattern to calculate a phase-intensity distribution profile in order to achieve a selected irradiation distribution in the body, and may further include applying the phase-intensity distribution profile. Applying a weighting may include deactivating at least one ultrasound source, and may include adjusting power level(s) of only a subset of ultrasound sources, for example by increasing power to a source having a high (or low) quality of acoustic coupling with the body, removing power to a source having a low coupling quality, or maintaining a constant total power of ultrasound. The method may further include displaying an ultrasound image. Applying a weighting may include determining whether a sufficient number of transducers having a sufficient quality of acoustic coupling to generate an ultrasound image of a selected quality, and may further include communicating an achievable image quality. Determining a quality of acoustic coupling may include determining the quality at a succession of points in time, and applying a weighting may include applying the weighting to ultrasonic data captured in the vicinity of each of these points in time.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of an ultrasound array.

FIG. 2 is a schematic of another ultrasound array.

FIG. 3 is a flow chart illustrating operation of an ultrasound array.

FIG. 4 is a flow chart illustrating weighting of ultrasonic data.

FIG. 5 is a schematic of another method of weighting ultrasonic data.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

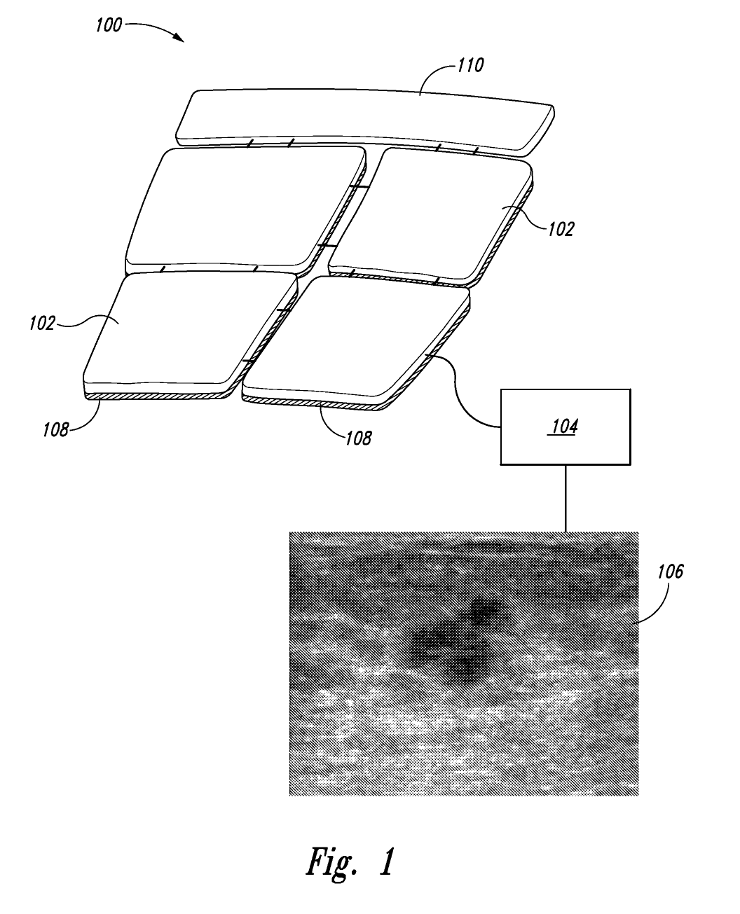

FIG. 1 is a schematic of an ultrasound array 100 configured for producing an ultrasound image. The array includes transducer elements 102 configured for contact with a body. Controller 104 monitors signals from transducer elements 102 indicating the quality of their acoustic coupling with the body, and applies a weighting to data received from the transducers 102 to produce an ultrasound image 106. In some embodiments, the quality of acoustic coupling at each transducer is determined by using the ultrasound transducer itself, either at the imaging frequency or at another frequency. Alternatively (or, in some embodiments, in addition), the quality of acoustic coupling may be determined by use of a separate contact sensor 108. For example, contact sensors 108 may be force measurement transducers, or they may be capacitive or resistive sensors. Ultrasound may enter the body from a source 110 (or plurality of sources) colocated with the transducer elements 102, or from a remote source or sources. In some embodiments including a plurality of sources, each source 110 is associated with a particular transducer element 102. In some embodiments, different transducer elements 102 may use different sound frequencies in order to differentiate between sources 110.

In a very simple embodiment, controller 104 may monitor coupling of transducer elements 102, and may simply omit imaging data received from a transducer not meeting a contact quality threshold from appearing in the ultrasound image. In other embodiments, there may be a more complicated relationship between contact quality and imaging data. For example, data may be “downgraded” and used only if there is no data available from a nearby transducer element 102 in better contact with the body. In one embodiment, only the best 90% (or 70% or 50% or 30%) transducer elements 102 (that is, the 90% of transducers 102 having the best acoustic contact with the body) are used in the image. The image may also include false color to identify more or less “reliable” areas. For example, all measured image data may be used to generate an ultrasound image, but pixels may be shaded in red in areas where contact with the body is poor, and in green in areas where it is good. In some cases, an ultrasonic image may have “holes” indicating that there was insufficient contact with the body in those areas, or the system may simply indicate that no image can be produced at all because of poor contact quality.

Monitoring of contact with the body may be static or dynamic in nature. For a single image of a non-moving body, it may be sufficient to determine quality of acoustic contact for each transducer once. For a longer imaging process and/or a body in motion, it may be preferable to dynamically monitor the contact with various transducers and to continuously adjust the resultant image, either by weighting the data and using “better” data more heavily, or by applying false color or similar cues to the image to alert the user to areas of better or worse image quality. Data weighting may be computational in nature (where pixels “count” more heavily when they are considered to be more reliable), or it may be accomplished by providing more power to ultrasound sources (e.g., transducers) that appear to have a better quality of contact with the underlying body.

In some embodiments, once the quality of acoustic coupling is measured, the system may attempt to remediate transducers having poor acoustic coupling. For example, the system may dispense additional ultrasound gel (or another acoustic coupling agent) to try to improve the quality of coupling at a transducer element 102 having a poor image quality rating, or it may adjust a contact force in the area having a poor contact rating.

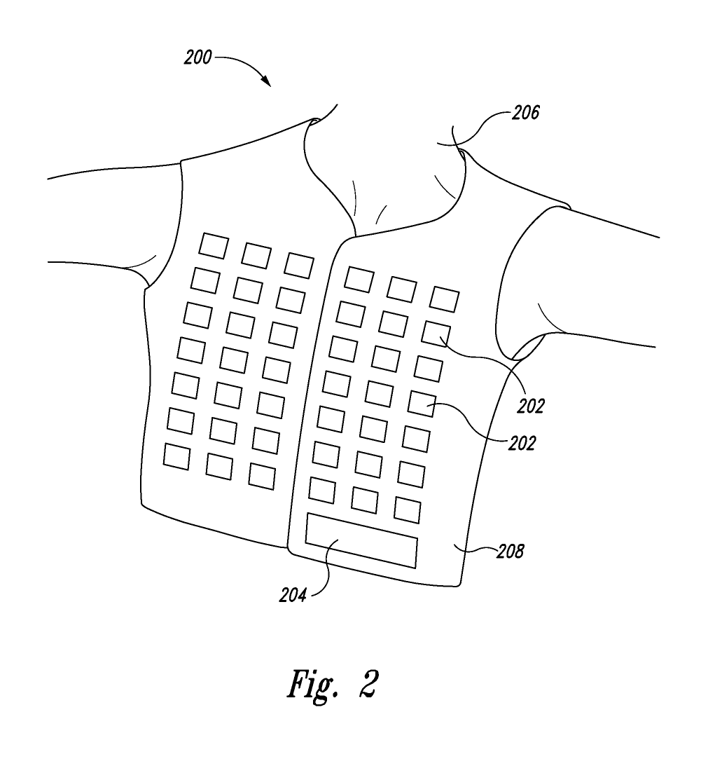

FIG. 2 is a schematic of another ultrasound array 200. As in FIG. 1, array 200 includes a plurality of ultrasound transducers 202, and controller 204 monitors the quality of acoustic coupling of transducers 202 with a body under examination 206. However, array 200 is not configured to produce a human-readable image such as that shown in FIG. 1. The array illustrated in FIG. 2 is configured for monitoring the condition of a human heart using a continuously-worn monitoring vest 208. Since the vest 208 is worn continuously during normal day-to-day activities (or possibly during a testing situation such as, for example, a cardiac stress test), transducers 202 are expected to vary their position and the quality of their acoustic contact with the body substantially over time.

Cardiac monitors such as the one illustrated in FIG. 2 typically use the Doppler effect to measure the velocity of blood through the aortic arch, using the relationship

$$f = \frac{2 v {f}_{0} cos θ}{c}$$

where v represents the blood speed, f₀ represents the ultrasound beam frequency, c represents the speed of ultrasound in blood (about 1.6×10³ m/s), and f represents the Doppler shift frequency. Since θ is relatively difficult to estimate, especially for a wearable ultrasound system like that illustrated in FIG. 2, it is preferable (but not required, as long as the angle of insonation is at least approximately known) to orient the beam to be as close as possible to parallel to the flow of blood. Since there are multiple transducers 202, controller 204 may also use their relative measurements to estimate the value of θ.

In use, controller 204 monitors the quality of acoustic coupling of transducers 202 with the body, and continuously calculates a best estimate for blood velocity. In some embodiments, data from all transducers 202 is used, but the transducers having the best acoustic contact with the body are weighted more heavily. In other embodiments, data from transducers 202 having poorer contact with the body is discarded. Blood velocity data may be recorded, e.g., for later review by a physician.

The transducer arrays described above may be used to obtain ultrasound data regarding a variety of different objects. In some embodiments, the array may be used to probe a living body or a human body, while in other embodiments, the array may be used to probe an article of manufacture (e.g., checking a casting for cracks before it is deployed). The transducer elements may be, for example, incorporated into a garment, which may be worn by a user in a testing situation and/or during daily activities.

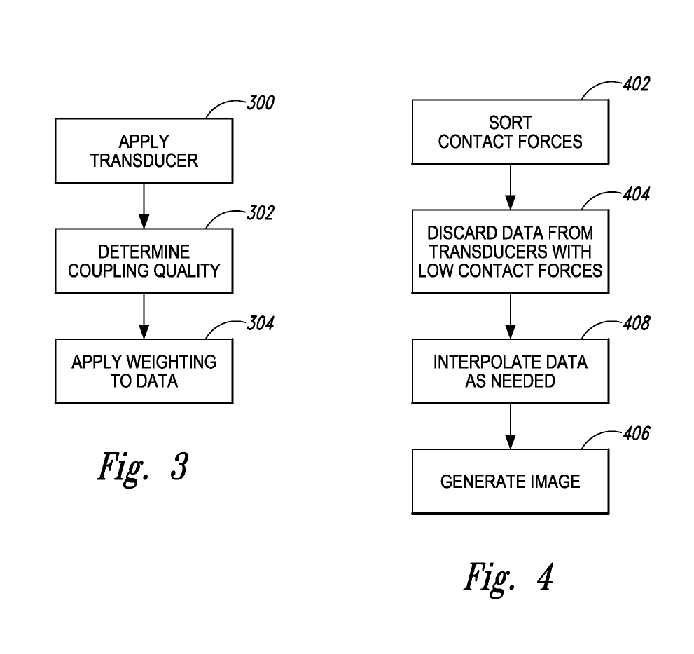

FIG. 3 is a flow chart illustrating use of an ultrasound array like the ones illustrated in FIG. 1 and FIG. 2. The method includes applying ultrasonic transducer elements to a body 300, determining a quality of acoustic coupling with the body at each tranducer element 302, and using the determined qualities of acoustic coupling to apply a weighting to ultrasonic data generated by each transducer element 304.

Determining a quality of acoustic coupling at (or in the immediate proximity of) each transducer element may include, for example, determining the magnitude of an echo or reflection from an exterior surface of the body, either at the imaging frequency or at a different frequency. A strong echo is typically associated with good coupling with the body. Alternatively, determining a coupling may include measuring contact with the body by measuring force, resistance, capacitance, or some other property. For example, a strain gage may be placed at the contact surface of a transducer element to measure the force between that element and the body, where a larger contact force is typically indicative of a better quality of acoustic coupling.

One an array of acoustic coupling data has been determined, it is used to apply a weighting. A simple method of weighting the data is illustrated in FIG. 4. The measured qualities (e.g., the contact forces) are sorted 402, and the bottom 30% are discarded 404. (Those of skill in the art will of course recognize that 30% is an arbitrary value, and that another threshold may equally well be substituted, either as a different percentage or as a different absolute value of the quality measure.) In some embodiments, explicitly discarding measurements from sites in poor contact will improve image reconstruction compared to the alternative of receiving a low or null signal due to poor coupling quality and assuming that this value represents an actual trough in the arriving ultrasonic wave. The remaining sensor data is used to generate an ultrasound image 406. In some embodiments, the 30% of transducers that are removed from the data create “holes” in the ultrasound image, while in other embodiments, data is interpolated 408 from neighboring transducers to show a complete image. In embodiments where the data is interpolated to produce a complete image, false color may be used to identify interpolated regions.

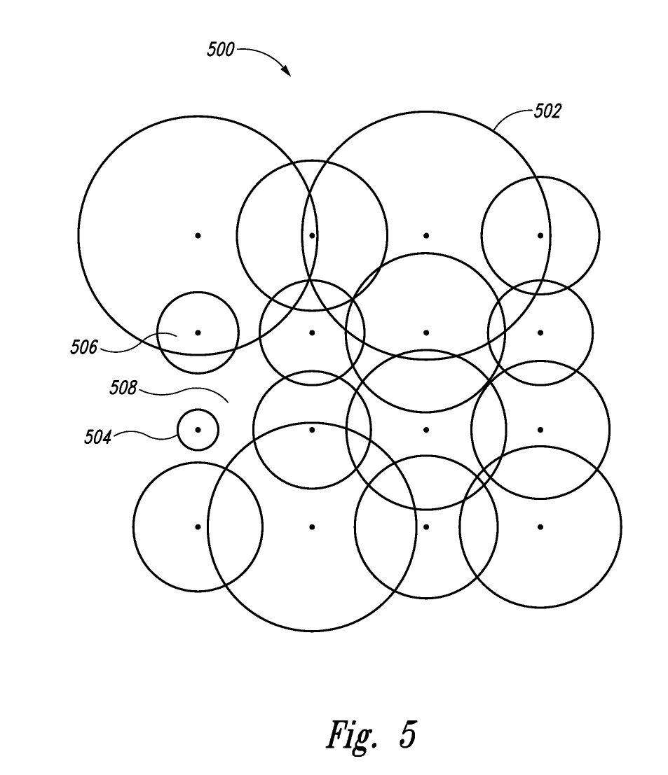

A slightly more complex method of data weighting is illustrated in FIG. 5. In this method, the quality of acoustic coupling at each transducer is used to determine a “spot size” for data from that transducer. FIG. 5 shows spot sizes 500 for a plurality of transducers. Those with relatively good coupling have large spots 502, while those with relatively poor coupling have smaller spots 504. In some embodiments of this type, spot sizes may go to zero for sufficiently poor acoustic coupling. When displaying an image, any given pixel is displayed using the ultrasonic measurement generated by the transducer having that pixel in its “spot.” If a pixel appears in multiple spots (as at point 506), it either uses an average of the values of the spots overlapping the pixel, or it uses the largest spot that overlaps it. No data is generated for pixels falling outside the spots (such as at point 508).

In some embodiments, determination of the acoustic coupling quality may be used to adjust one or more power levels for ultrasound source(s). This may include, for example, increasing power to sources having good contact, or decreasing (or turning off entirely) sources having poor contact. This may include increasing power to sources having poor contact so as to maintain or equalize a desired level of ultrasonic coupling into the body. In some embodiments, the determined quality of acoustic coupling for an ultrasonic source may be used to determine a measure of how much of its output actually couples into the body; this can then be used within data analysis algorithms by replacing source distributions based on emitted power by ones based on coupled power.

In some embodiments, knowledge of the spatial profile of coupling quality for an array of ultrasonic transducers can be used in operation of a phased array. The activation of individual sources can be based upon the spatial pattern of those having sufficient coupling quality, and the power delivered to each source can depend upon its coupling quality in order to achieve a desired spatial pattern of body-coupled ultrasonic power. Similarly, the spatial pattern of the coupling quality can also be used in determining the reception properties of a phased array. Array elements having poor coupling quality can be deleted from the antenna pattern, and received ultrasonic signals at a location can be divided by the coupling quality to provide a better measure of the ultrasonic signal arriving at the surface of the body.

In a general sense, those skilled in the art will recognize that the various aspects described herein which can be implemented, individually or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes or devices described herein, or a microprocessor configured by a computer program which at least partially carries out processes or devices described herein), electrical circuitry forming a memory device (e.g., forms of random access memory), or electrical circuitry forming a communications device (e.g., a modem, communications switch, or optical-electrical equipment). Those having skill in the art will recognize that the subject matter described herein may be implemented in an analog or digital fashion or some combination thereof

Those having skill in the art will recognize that the state of the art of circuit design has progressed to the point where there is typically little distinction left between hardware and software implementations of aspects of systems. The use of hardware or software is generally a design choice representing tradeoffs between cost, efficiency, flexibility, and other implementation considerations. Those having skill in the art will appreciate that there are various vehicles by which processes, systems or other technologies involving the use of logic or circuits can be effected (e.g., hardware, software, or firmware), and that the preferred vehicle will vary with the context in which the processes, systems or other technologies are deployed. For example, if an implementer determines that speed is paramount, the implementer may opt for a mainly hardware or firmware vehicle. Alternatively, if flexibility is paramount, the implementer may opt for a mainly software implementation. In these or other situations, the implementer may also opt for some combination of hardware, software, or firmware. Hence, there are several possible vehicles by which the processes, devices or other technologies involving logic or circuits described herein may be effected, none of which is inherently superior to the other. Those skilled in the art will recognize that optical aspects of implementations may require optically-oriented hardware, software, and or firmware.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a transducer” should typically be interpreted to mean “at least one transducer”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two transducers,” or “a plurality of transducers,” without other modifiers, typically means at least two transducers). Furthermore, in those instances where a phrase such as “at least one of A, B, and C,” “at least one of A, B, or C,” or “an [item] selected from the group consisting of A, B, and C,” is used, in general such a construction is intended in the sense one having skill in the art would understand the convention (e.g., any of these phrases would include but not be limited to systems that have A alone, B alone, C alone, A and B together, A and C together, B and C together, or A, B, and C together). It will be further understood by those within the art that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An ultrasound array, comprising:
a plurality of transducer elements configured for contact with a body; and
a controller configured to determine a quality of acoustic coupling with the body at each transducer element and to use the determined coupling qualities to apply a weighting to ultrasonic data received from the plurality of transducer elements.

2. The ultrasound array of claim 1, further comprising an ultrasound source.

3. The ultrasound array of claim 1, further comprising a plurality of ultrasound sources.

4. The ultrasound array of claim 3, wherein each ultrasound source is associated with a corresponding member of the plurality of transducer elements.

5. The ultrasound array of claim 1, wherein the plurality of transducer elements are configured for contact with a living body.

6. The ultrasound array of claim 1, wherein the plurality of transducer elements are configured for contact with a human body.

7. The ultrasound array of claim 1, wherein the plurality of transducer elements are incorporated into a garment.

8. The ultrasound array of claim 1, wherein determining the quality of acoustic coupling includes determining magnitude of an echo or reflection from an exterior surface of the body.

9. The ultrasound array of claim 1, wherein determining the quality of acoustic coupling includes determining magnitude of a signal passing through the body.

10. The ultrasound array of claim 1, wherein determining a quality of coupling includes applying an ultrasound signal at a first frequency to determine a set of coupling qualities, and wherein applying a weighting includes applying the weighting to ultrasonic data gathered at a second frequency different from the first frequency.

11. The ultrasound array of claim 1, further comprising a display configured to display an ultrasound image using the weighted ultrasonic data.

12. The ultrasound array of claim 1, wherein applying a weighting includes using ultrasonic data from a selected subset of the plurality of transducer elements.

13. The ultrasound array of claim 12, wherein applying a weighting includes applying a weighting to one element of the selected subset using contact quality data from another element of the selected subset.

14. The ultrasound array of claim 1, wherein applying a weighting includes discarding null signals from transducers having a relatively poor quality of acoustic coupling with the body.

15. The ultrasound array of claim 1, wherein applying a weighting includes determining whether a sufficient number of transducers have a sufficient quality of acoustic coupling to generate an ultrasound image of a selected quality.

16. The ultrasound array of claim 15, wherein determining whether a sufficient number of transducers have a sufficient quality of acoustic coupling includes signaling that the ultrasound image of the selected quality cannot be produced.

17. The ultrasound array of claim 15, wherein determining whether a sufficient number of transducers have a sufficient quality of acoustic coupling includes producing an ultrasound image only after determining that the selected quality of ultrasound image can be achieved.

18. The ultrasound array of claim 1, wherein the transducers are configured to measure a Doppler shift frequency.

19. The ultrasound array of claim 1, wherein the array is configured to respond to the determined quality by adjusting a quantity of coupling fluid between a transducer and the body.

20. The ultrasound array of claim 1, wherein the array is configured to respond to the determined quality of acoustic coupling by adjusting a contact force of the transducer.

21. The ultrasound array of claim 1, wherein determining a quality of acoustic coupling with the body at each transducer element includes determining the quality at a succession of points in time.

22. The ultrasound array of claim 21, wherein applying a weighting includes applying the weighting to ultrasonic data captured in the vicinity of each point in time.

23. An ultrasound array, comprising:
a plurality of ultrasound sources configured for contact with a body;
a plurality of ultrasound receivers configured to receive ultrasound from the plurality of ultrasound sources; and
a controller configured to determine a quality of acoustic coupling with the body at each ultrasound source and to use the determined qualities of acoustic coupling to apply a weighting to a power level of each of the plurality of ultrasound sources.

24. The ultrasound array of claim 23, wherein applying a weighting includes determining an in-contact array pattern for the ultrasound sources, and further includes using the determined in-contact array pattern to calculate a phase-intensity distribution profile selected to deliver a selected irradiation distribution in the body.

25. The ultrasound array of claim 23, wherein applying a weighting includes adjusting a power level of a selected subset of the plurality of ultrasound sources.

26. The ultrasound array of claim 25, wherein adjusting a power level includes increasing power to an ultrasound source having a superior quality of acoustic coupling with the body compared to another ultrasound source.

27. The ultrasound array of claim 25, wherein adjusting a power level includes increasing power to an ultrasound source having an inferior quality of acoustic coupling with the body compared to another ultrasound source.

28. The ultrasound array of claim 25, wherein adjusting a power level includes removing power to an ultrasound source having an inferior quality of acoustic coupling with the body compared to another ultrasound source.

29. The ultrasound array of claim 25, wherein adjusting a power level includes maintaining a constant total energy of ultrasound coupled into the object by the plurality of ultrasound sources.

30. The ultrasound array of claim 25, wherein adjusting a power level includes maintaining a constant total energy of ultrasound coupled into the object by a member of the plurality of ultrasound sources.

31.-34. (canceled)

35. The ultrasound array of claim 23, wherein the plurality of ultrasound sources are configured to provide ultrasound at a first frequency for determining a quality of acoustic coupling, and at a second frequency different from the first frequency for producing an ultrasound image.

36. (canceled)

37. The ultrasound array of claim 23, wherein applying a weighting to a power level of each of the plurality of ultrasound sources includes deactivating at least one ultrasound source.

38. The ultrasound array of claim 23, wherein applying a weighting to a power level of each of the plurality of ultrasound sources includes adjusting a total power to a level that provides a selected image quality.

39. (canceled)

40. The ultrasound array of claim 23, wherein the ultrasound receivers are configured to measure a Doppler shift frequency.

41. The ultrasound array of claim 23, wherein the array is configured to respond to the determined quality of acoustic coupling by adjusting a quantity of coupling fluid between an ultrasound receiver and the body.

42. The ultrasound array of claim 23, wherein the array is configured to respond to the determined quality of acoustic coupling by adjusting a quantity of coupling fluid between an ultrasound source and the body.

43. The ultrasound array of claim 23, wherein the array is configured to respond to the determined quality of acoustic coupling by adjusting a contact force between an ultrasound receiver and the body.

44. The ultrasound array of claim 23, wherein the array is configured to respond to the determined quality of acoustic coupling by adjusting a contact force between an ultrasound source and the body.

45.-46. (canceled)

47. A ultrasound method, comprising:
applying a plurality of ultrasonic transducer elements to a body;
determining a quality of acoustic coupling with the body at each transducer element; and
using the determined qualities of acoustic coupling to apply a weighting to ultrasonic data generated by each transducer element.

48.-71. (canceled)

72. An ultrasound method, comprising:
applying a plurality of ultrasound sources to a body;
applying a plurality of ultrasound receivers to the body, the ultrasound receivers being configured to receive ultrasound from at least one of the ultrasound sources;
determining a quality of acoustic coupling with the body at each ultrasound source; and
using the determined qualities of acoustic coupling to apply a weighting to a power level of each of the ultrasound sources.

73.-87. (canceled)

88. An ultrasound array, comprising:
a plurality of transducer elements configured for contact with a body; and
a controller configured to determine a quality of acoustic coupling with the body at each transducer element and to use the determined qualities of acoustic coupling to deactivate transducer elements not meeting a threshold quality of acoustic coupling.

89.-99. (canceled)
